(19) United States
(12) Patent Application Publication
(10) Pub. No.: US 2004/0147924 A1
(43) Pub. Date: Jul. 29, 2004

(54) Bipolar stimulating electrode

(76) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 10/759,791

(22) Filed: Jan. 16, 2004

(30) Foreign Application Priority Data
Jan. 20, 2003 (DE) 103 02 319.4

(51) Int. Cl. A61N 1/04

(52) U.S. Cl. 606/48

(57) ABSTRACT

An implantable bipolar stimulating electrode (1) has a cathode (3) to be fixed in the heart (2) and an anode (4), which is set at a distance to the cathode and which is arranged near or on the connector (5) used to connect the stimulating electrode 1 to a cardiac pacemaker (7). In the working position the anode (4) is adapted to be located outside of the vein (8), through which the supply line to the cathode (3) is guided. The entire electrode and especially also the region near the cathode (3) can therefore be formed with corresponding flexibility.

### BACKGROUND

The invention relates to an implantable bipolar stimulating electrode with a cathode to be fixed in the heart, with an anode, which forms a second pole and which is at a distance to the cathode, and with a connector for connecting the stimulating electrode to an implantable cardiac pacemaker. At the cardiac pacemaker, this connector is located outside of the vein, which leads to the heart and through which the stimulating electrode is guided to the working position.

A stimulating electrode of this type is known, e.g., from CH 653 559 A5 and from U.S. Pat. No. 5,545,205.

For the bipolar stimulating electrode described in CH 653 559 A5, as well as for comparable stimulating electrodes, the neutral pole, that is, the anode, is always located at a proximal distance of approximately 10 mm-40 mm from the electrode head, which forms the cathode. Because both the cathode and also the anode consist of solid, electrically conductive material, usually metal, and the neutral electrode, that is, the anode, always has a significantly greater surface area than the cathode, usually formed by a metallic tube, it cannot be prevented that the distal part of such a bipolar implantable stimulating electrode with a length of approximately 20-40 mm or even somewhat more has a certain stiffness.

Also for the arrangement described in U.S. Pat. No. 5,545,205, the stimulating electrode in the working position is located so close to the electrode head that it comes to lie within the heart. For this arrangement, this also produces a certain stiffness in the stimulating electrode.

Relatively stiff stimulating electrodes have the tendency to not always follow the movements triggered by the heart contraction and therefore cause a certain amount of irritation to the heart inner wall. Therefore, fibrotic tissue forms between the electrode head and the tissue of the heart inner wall to be stimulated. In turn, this has the consequence that the electrode head is lifted from the tissue to be stimulated. Because the stimulation threshold changes with the square of the distance between the electrode head and the tissue to be stimulated, an increase of the stimulation threshold must be taken into account.

The corresponding problem is intensified if two stimulating electrodes, one of which is anchored, e.g., in the vestibule and the other in the ventricle, extend out from a cardiac pacemaker, for which bipolar stimulating electrodes are preferred.

Due to the adjacent anode at a distance to the cathode, the stimulating electrode is also provided with a corresponding supply line up to the anode, so that the stimulating electrode has reduced flexibility relative to a unipolar electrode.

U.S. Pat. No. 3,915,174 describes a stimulating electrode, for which, in the working position, the anode is located outside of the heart but inside of the veins leading to the heart. Thus, in this case a corresponding stiffness of the stimulating electrode must also be taken into account over the region leading up to this anode.

### SUMMARY

Therefore, the object is to create a bipolar stimulating electrode of the type mentioned in the introduction, which features a supply line of high flexibility up to the cathode.

To achieve this object, the stimulating electrode defined in the introduction with the connector for connecting to the cardiac pacemaker is characterized in that the anode is arranged near or on the connector on its side facing away from cardiac pacemaker, such that in the working position, it is located between the connector and the entrance into the vein.

Surprisingly, the anode is thus arranged at a considerably greater distance to the cathode, so that from this anode outwards, the supply line to the cathode can have a high flexibility and also its dimensions can be reduced, because a supply line to the anode in this region is no longer required. In addition, the anode which results in the additional stiffness is not found in the interior of the heart, because this can now be located outside of the vein close to the cardiac pacemaker, where there is sufficient room for it, even if several stimulating electrodes extend out from the cardiac pacemaker.

For example, the anode can be arranged directly adjacent to a socket of the connector. Such sockets, also acting as kink protection, enclose a large part of the connector in an insulating way and only expose the connector pin, which can be inserted into a corresponding connector on the cardiac pacemaker. The anode of the bipolar stimulating electrode can thus be arranged directly adjacent to this socket surrounding and insulating the connector on the outside, whereby after the implantation it is located at a position, which is still outside of the vein, through which the stimulating electrode is guided into the heart.

Through the arrangement according to the invention near or on the connector of the cardiac pacemaker, there results an electrode shaft having high flexibility, which leads to less mechanical irritation of the heart and thus to lower stimulation thresholds. This also permits a smaller shaft diameter and thus also access through smaller veins. This is an advantage, above all, if two-chamber stimulation is desired, for which one stimulating electrode is to be anchored in the vestibule and another stimulating electrode is to be anchored in the ventricle. Furthermore, there are no insulation problems, like those which must be solved for previously relatively long parallel supply lines to the cathode and anode. In addition, the material consumption is less, because the supply line to the anode is shorter, so that production costs are also reduced. Finally, there results higher reliability, because a shorter supply line to the anode also has a lower risk of breaking.

Another configuration of the invention of considerably more significance is provided in that a receiver part, a coupling, or an adapter or the like for connecting to the proximal end of the stimulating electrode is on the side of the anode facing away from the cardiac pacemaker and the connector. Therefore, the anode is also located in a region adjacent to the connector to the cardiac pacemaker, but the supply line to the heart can be connected over another connector, so that if necessary, an exchange is also possible. Above all, if a pacemaker is replaced, any already implanted, previously unipolar electrodes can be converted into a bipolar electrode by connecting the end of this unipolar electrode facing the cardiac pacemaker to the receiver part, the coupling, or the adapter, which leads to the anode. In this way, a unipolar stimulating electrode with a non-standard connector can also be converted into a bipolar stimulating electrode according to the invention by removing the previous connector on this unipolar electrode and connecting the coil or the like, after stripping the insulation, to an adapter with a coupling, as is known from DE PS 39 06 598.

The anode can be a bare metal sleeve on the outside, especially made from platinum, platinum-iridium, or a similar material with good conductive properties. Because in the working position it is located at a location outside of the vein leading to the heart, it can be dimensioned large accordingly, without negatively affecting the flexibility of the actual stimulating electrode.

The anode can also be a perforated anode, wherein it can be arranged as a coil or sleeve within a perforated silicon jacket. The anode can be further configured as a coil made from bare wire.

In order to stimulate the heart several times, several electrodes can also extend from one cardiac pacemaker.

However, it is also possible for at least two anodes to be arranged insulated from each other on a common carrier one behind the other and to be allocated to at least two cathodes that can be attached in the heart. Thus, power can be supplied with an example four-pole connector and a single carrier for several anodes of a bipolar arrangement, which can be arranged according to the invention adjacent to the corresponding connector in a space-saving way one behind the other.

Above all, for the combination of individual or several features and measures described above, a bipolar implantable stimulating electrode is produced, whose region running through a vein to the heart and whose end anchored in the heart can exhibit high flexibility like a unipolar electrode, so that the dimensions can also be reduced, without losing the advantage of the bipolar configuration, so that several such bipolar stimulating electrodes can also extend from a common cardiac pacemaker and can work with different stimulating effects.

### BRIEF DESCRIPTION OF THE DRAWINGS

In the following, embodiments of the invention are described in more detail with reference to the drawing. Shown in partially schematic illustration are:

FIG. 1 is a view of a cardiac pacemaker with a stimulating electrode according to the invention, which is guided into the ventricle of a heart, wherein the anode of this bipolar stimulating electrode is directly adjacent to the connector of the cardiac pacemaker,

FIG. 2 is a view corresponding to FIG. 1, for which two bipolar stimulating electrodes according to the invention extend from the cardiac pace-maker, one of which is anchored in the ventricle and the other in the vestibule of the heart, wherein each of these stimulating electrodes has an anode adjacent to a connector to the cardiac pacemaker, wherein the two connectors and anodes are offset relative to each other for reasons of space, so that they have different distances to the cardiac pacemaker,

FIG. 3 is an enlarged scale view of a bipolar stimulating electrode according to the state of the art, for which the anode is arranged at a distance to the connector and adjacent to the cathode,

FIG. 4 is a view similar to FIG. 3 of a stimulating electrode according to the invention for which the anode, formed as a bare coil, is arranged in direct connection to a socket of the connector,

FIG. 5 is a view of a stimulating electrode, for which the coil-shaped anode is partially enclosed,

FIG. 6 is a view of a stimulating electrode, for which the anode is formed as a bare sleeve,

FIG. 7 is a view of a stimulating electrode, for which the anode is formed as a perforated anode, wherein the actual conductive part is arranged as a coil or sleeve within a perforated silicon part,

FIG. 8 is a view of an embodiment of the invention, for which the anode to an adapter is provided with a receiver part, in which a connector of the actual stimulating electrode, e.g., a unipolar electrode, fits,

FIG. 9 is a view of an embodiment of the invention similar to FIG. 8, for which on the part with the anode at its end facing away from the connector there is a coupling for connecting the insulation-stripped end of the electrode, in particular, of a unipolar electrode,

FIG. 10 is a view of an arrangement with a modified adapter, for which an insulation-stripped end of the particularly unipolar stimulating electrode can be inserted through the part with the anode and through a channel located in the interior up to a hollow connector pin and can be pressed there according to DE PS 39 06 598, and

FIG. 11 is a view of a stimulating electrode according to the invention with two anodes, which are arranged on a common carrier one behind the other, which are insulated from each other, and which are allocated to two cathodes attached after a fork, so that two bipolar stimulating actions can be performed at different locations in the heart.

### DETAILED DESCRIPTION OF THE PREFERRED EMBODIMENTS

A stimulating electrode designated as a whole with 1 has a cathode 3 to be fixed in a given manner in the heart 2, e.g., in the ventricle or also in the vestibule, thus forming a first pole of the bipolar stimulating electrode.

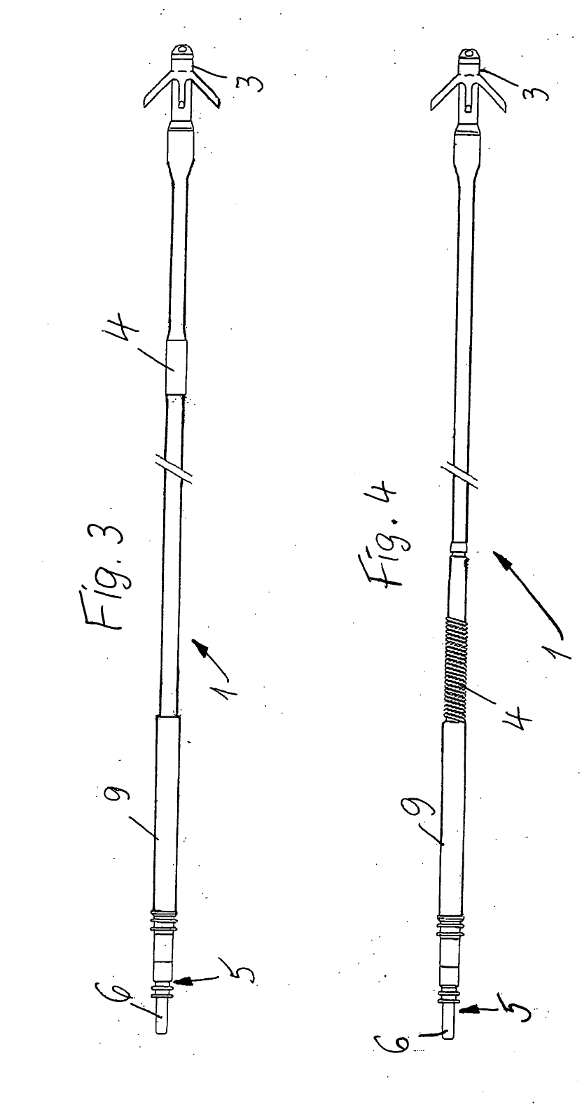

The stimulating electrode 1 further has an anode 4, which forms a second pole, which is set at a distance to the cathode 3, and which has a relatively small distance to the cathode 3 in the state of the art according to FIG. 3, and therefore produces a relatively stiff stimulating electrode 1.

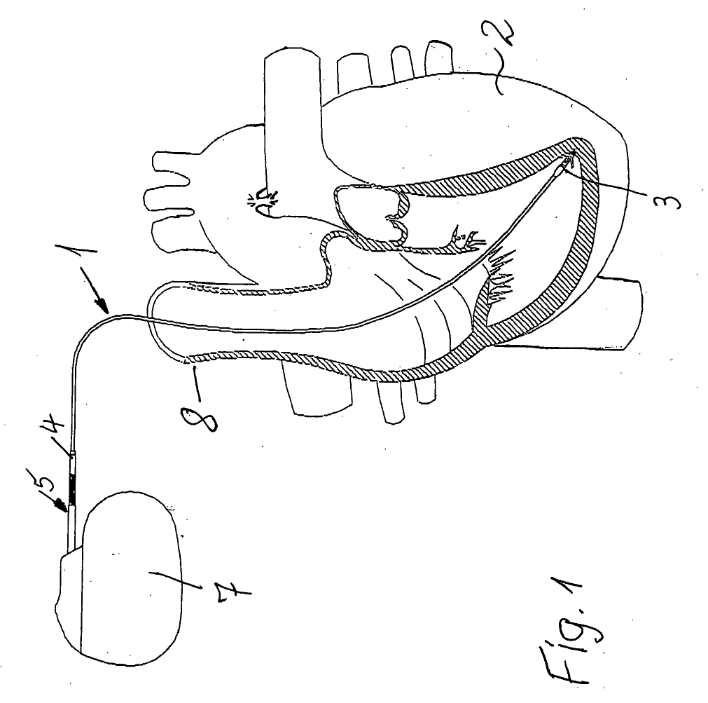

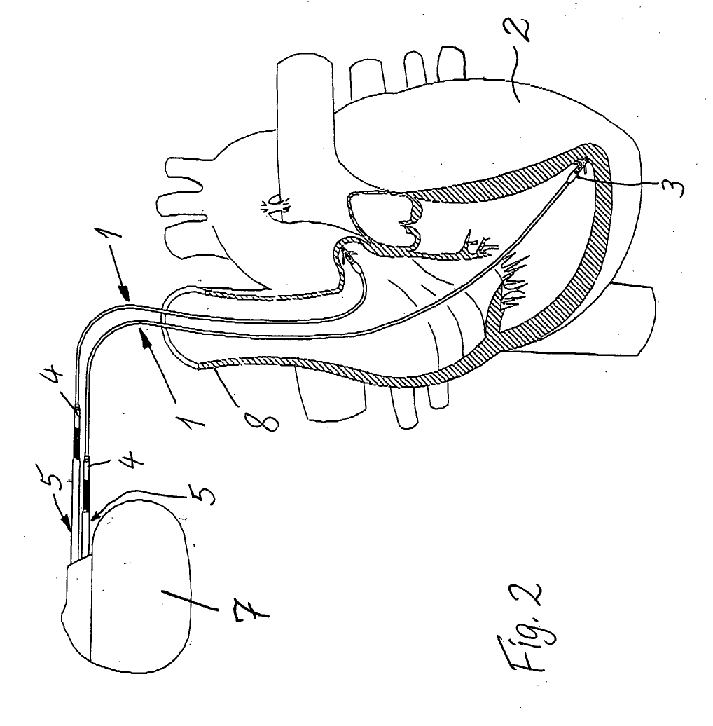

The stimulating electrode 1 in accordance with the invention further includes a connector 5, whose connector pin 6 fits into a corresponding receptacle or bushing in an implantable cardiac pacemaker 7, so that the stimulating electrode 1 can be connected to the cardiac pacemaker 7 with the help of this connector 5. In this way, this connector 5 according to FIGS. 1 and 2 is located outside of the vein 8, which leads to the heart 2, of which only a short piece close to the heart 2 is shown in FIGS. 1 and 2, and through which the stimulating electrode 1 is guided to the working position.

According to FIGS. 1 and 2, as well as according to the FIGS. 4-10, the anode 4 is arranged near or on the connector 5 on its side facing away from the cardiac pacemaker 7 such that in the working position, it is located between the connector 5 and the entrance into the vein 8. In this way, the anode 4 in the working position is arranged outside of the vein 8, so that the part of the stimulating electrode 1 running from the anode 4 to the cathode 3 can be made thinner and more flexible, because in this region a supply line to the anode 4 is no longer necessary. Thus, the bipolar stimulating electrode according to the invention can also be inserted into relatively thin veins 8.

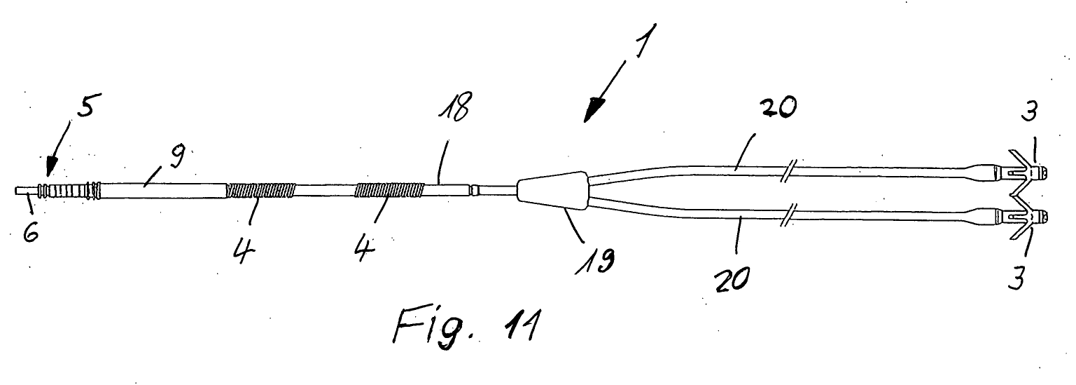

While the cathode 3 for stimulating electrodes 1 according to the state of the art, as shown in FIG. 3, are arranged relatively close to the cathode 3 and thus their supply line must run over nearly the entire length of the stimulating electrode 1, the various embodiments of the anode 4 according to FIGS. 4-10 are each arranged directly adjacent to a socket 9 of the connector 5. This can be seen especially clearly in FIGS. 4, 8, and 9, in which the anode 4, as in the embodiment according to FIG. 10, is formed as a coil made from bare wire.

This arrangement of the anode 4 has an especially advantageous effect in cases, for which several stimulating electrodes 1 extend out from a cardiac pacemaker 7 and generate different stimulating effects, so that the housing of the cardiac pacemaker would not be usable as an anode.

In this way, the connector 5 and thus also the anodes 4 for such a multiple arrangement of stimulating electrodes 1 can be offset relative to each other on the same cardiac pacemaker 7, such that the connector 5 or its connections have different lengths.

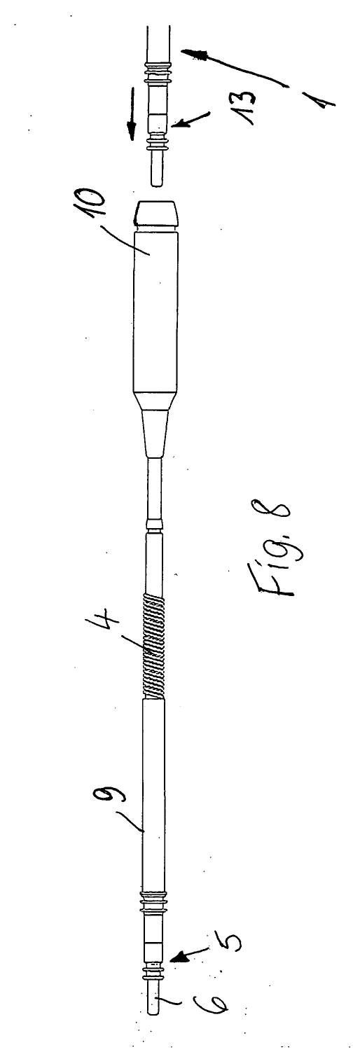

FIGS. 8-10 show special embodiments. In these cases, on the side of the anode 4 facing away from the cardiac pacemaker 7 and the connector 5, there is a receiver part 10 or a coupling 11 or an adapter 12 or the like for connecting to the proximal, insulation-stripped end of the stimulating electrode 1, i.e., the stimulating electrode 1 has a gap between the anode 4 and the cathode 3, which can be connected at a later time.

FIG. 8 shows an embodiment with a receiver part 10, in which fits a typical, standardized connector 13, which could also be inserted directly into a cardiac pacemaker 7. This arrangement allows already implanted, previously unipolar functioning stimulating electrodes 1 to be made into bipolar stimulating electrodes 1 by inserting the anode 4 with the receiver part 10 when the cardiac pacemaker 7 is exchanged.

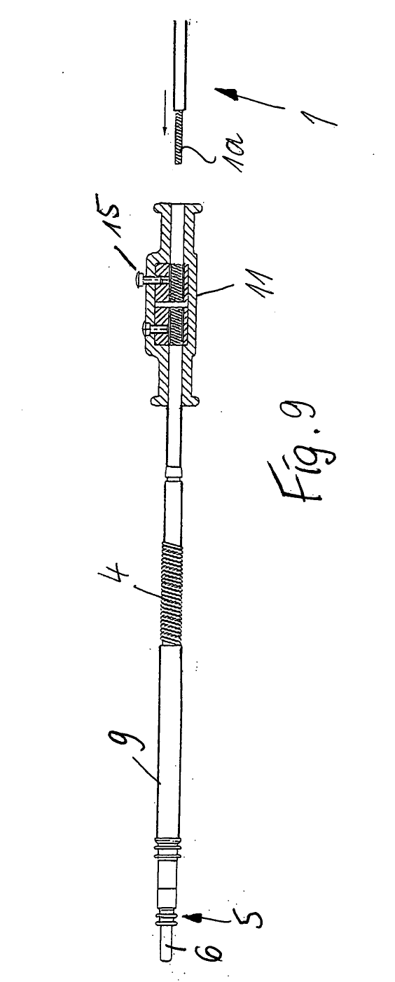

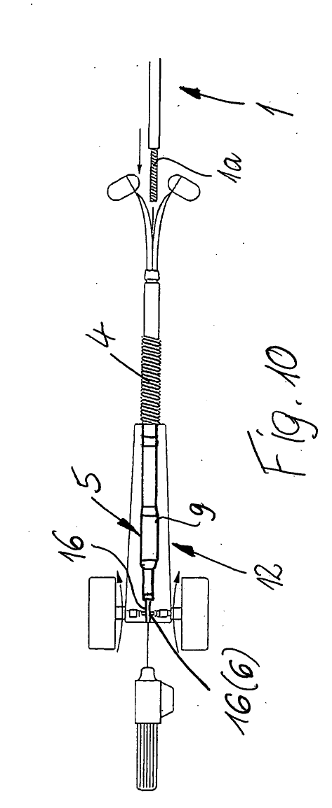

An analogous situation applies for the embodiment according to FIG. 9, for which the coupling 11 is connected on one side to an electrode part, which leads to the anode 5, while the insulation-stripped end la of the stimulating electrode 1 can be inserted from the other side and can be fixed with clamping screws 15, and then can be inserted as the connector pin 6 into the cardiac pace-maker 7.

FIG. 10 shows a third possibility for converting an originally unipolar stimulating electrode into a bipolar stimulating electrode 1, for which the insulation-stripped end of the stimulating electrode is guided through an inner open channel of the anode 4 up to a hollow connector pin 16 in the interior, which is then pressed together, as is known from U.S. Pat. No. 5,050,602 and DE PS 39 06 598.

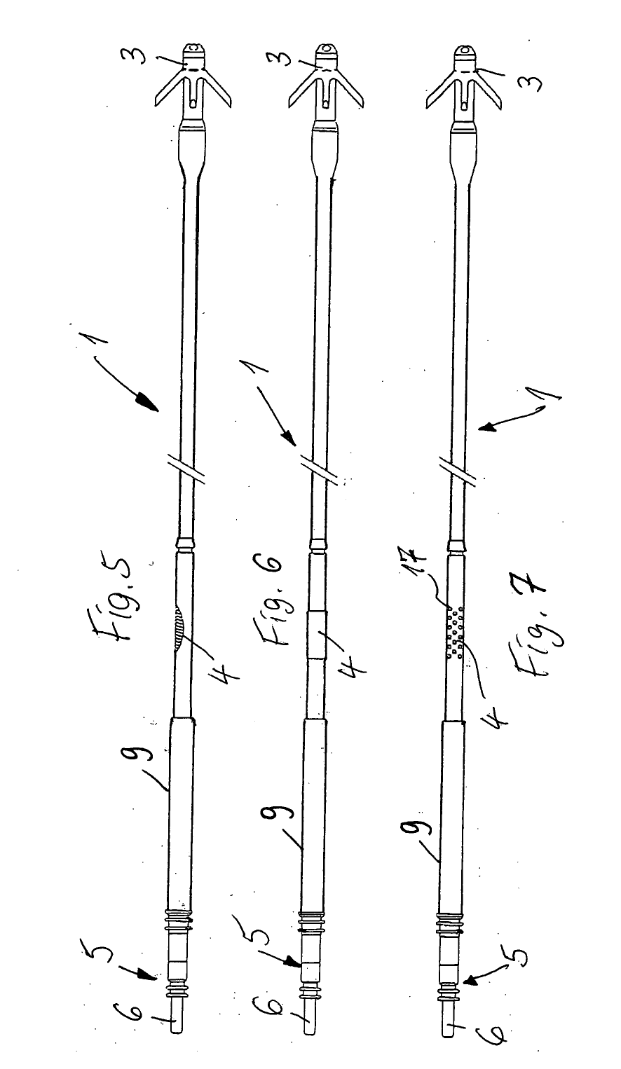

While it has already been mentioned that the anode 4 can be a coil, FIG. 6 shows the possibility of manufacturing the anode 4 as an exterior, bare metal sleeve, especially made from platinum, platinum-iridium, or a similar material with good conductive properties.

FIG. 7 shows a solution, for which the anode 4 is a so-called perforated anode, wherein it is arranged as a coil or sleeve within a perforated silicon jacket 17.

In FIG. 11, a stimulating electrode 1 is shown, which could be called a multiple electrode. Adjacent to a connector 5, which has four poles in this case, two anodes 4 insulated from each other are arranged one behind the other on a common carrier 18 and allocated to two cathodes 3 that can be attached in the heart 2, wherein behind the carrier 18 a fork 19 can be seen, which comes to lie like the anodes 4 before the entrance into the corresponding veins 8 in the working position, so that the actual supply lines 20 to the cathodes 3 can be individually “laid” projecting away from the fork 19. This permits a similar arrangement and stimulation effect, as explained with reference to FIG. 2, wherein, however, a single connector 5 with correspondingly many poles can be inserted into the cardiac pacemaker 7.

The anodes 4 are formed as coils in the embodiment according to FIG. 11, but they can also have other shapes, e.g., analogous to FIGS. 5, 6, or 7.

The implantable bipolar stimulating electrode 1 has a cathode 3 to be fixed in the heart 2 and an anode 4, which is set at a distance to the cathode and which is arranged near or on the connector 5 used to connect the stimulating electrode 1 to a cardiac pacemaker 7, so that the anode 4 in the working position is located outside of the vein 8, through which the supply line is guided to the cathode 3. The entire electrode and particularly also the region near the cathode 3 can therefore be formed with corresponding flexibility.

1. Implantable bipolar stimulating electrode (1), comprising a cathode (3) adapted to be fixed in the heart (2), an anode (4) forming a second pole and at a distance to the cathode (3), and a connector (5) for connecting the stimulating electrode (1) to an implantable cardiac pacemaker (7), wherein at the cardiac pacemaker the connector (5) is adapted to be located outside of a vein which leads to the heart (2) and through which the stimulating electrode (1) is guided to the working position, the anode (4) is arranged near or on the connector (5) on a side facing away from the cardiac pacemaker (7) such that in the working position the anode(4) is located between the connector (5) and an entrance into the vein (8).

2. A stimulating electrode according to claim 1, wherein the anode (4) is arranged directly adjacent to a socket (9) of the connector (5).

3. A stimulating electrode according to claim 1, wherein on the side of the anode (4) facing away from the cardiac pacemaker (7) and the connector (5), there is at least one of a receiver part (10), a coupling (11), or an adapter (12) for connecting to a proximal end of the stimulating electrode (1).

4. A stimulating electrode according to claim 1, wherein the anode (4) is formed as a bare metal sleeve on an outside thereof, made from at least one of platinum, platinum-iridium, or a material with good conductive properties.

5. A stimulating electrode according to claim 1, wherein the anode (4) is a perforated anode, and is arranged as a coil or sleeve within a perforated silicon jacket (17).

6. A stimulating electrode according to claim 1, wherein the anode (4) is formed as a coil made from bare wire.

7. A stimulating electrode according to claim 1, wherein at least a second anode (4) that is insulated from the first anode and located adjacent to the connector (5) is provided, the anodes are arranged on a common carrier (18) one behind the other and are allocated to at least two cathodes (3) that can be attached in the heart (2).

8. A stimulating electrode according to claim 7, wherein there is a fork (19) for the supply lines (20) to the cathodes (3) at an end of the common carrier (18) for the anodes (4).
